(19) United States
(12) Patent Application Publication
(10) Pub. No.: US 2006/0287639 A1
(43) Pub. Date: Dec. 21, 2006

(54) Safety fluid transfer cannula

(75) Inventor: Fraser R. Sharp, Vancouver (CA)

Correspondence Address: NIXON & VANDERHYE, PC, 901 NORTH GLEBE ROAD, 11TH FLOOR, ARLINGTON, VA, 22203, US

(73) Assignee: Inviro Medical Devices Ltd., Bridgetown (BB)

(21) Appl. No.: 11/449,771

(22) Filed: Jun. 9, 2006

Related U.S. Application Data
(63) Continuation-in-part of application No. 11/346,302, filed on Feb. 3, 2006.
(60) Provisional application No. 60/690,520, filed on Jun. 15, 2005.

(51) Int. Cl. A61B 19/00

(52) U.S. Cl. 604/415

(57) ABSTRACT

A cannula for transferring fluid relative to a vial or intravenous port having an elastomeric membrane includes: a cannula body having first and second opposite ends; the first end terminating in a tip for penetrating the elastomeric membrane; the body having a passage opening through the second end and extending within the cannula body towards the first end, the passage opening through at least one horizontal-oriented port through a side surface of the cannula body thereby to enable flow of fluid along the passage and between the opening through the second end and the side port. An axial width of the port is smaller than a thickness dimension of a vial through which the cannula tip is to be inserted. A related method is also described.

### CROSS-REFERENCES TO RELATED APPLICATIONS

This application is a continuation-in-part of application Ser. No. 11/346,302, filed Feb. 3, 2006, which in turn claims priority from U.S. Provisional Patent Application Ser. No. 60/690,520, filed Jun. 15, 2005.

### BACKGROUND OF THE INVENTION

The present invention relates to a fluid aspiration and injection cannula or syringe barrel extension having safety features to preclude needle stick injuries and particularly relates to an aspiration/injection cannula particularly useful for withdrawing fluid from one or more standard vials, e.g., a medication vial into a standard hypodermic syringe and/or for injecting fluid into a similar vial or other containers or access ports such as the port of an intravenous (IV) line in a manner permitting safe transfer of fluid minimizing or eliminating the potential for accidental needle stick injuries. In the unlikely event that a skin penetration did occur with the subject cannula, even if it is contaminated, the likelihood of transmission of disease is reduced when compared to a standard hollow sharp metal needle.

The subject cannula may be a stand alone and removable device which can be fitted to a standard syringe or may be manufactured as an integral portion of a syringe barrel or a syringe barrel extension. This latter embodiment would constitute a non-removable syringe barrel extension with a penetrating tip having one or more features in common with the disclosed removable cannula.

The current healthcare work place, both in the hospital and in the home, offers many professional and personal safety challenges for healthcare workers. The increasing age of patients, the more complex nature of diseases and the incidence of serious infectious diseases such as HIV and Hepatitis all contribute to increased personal risk and demands for the healthcare worker. The diminishing number of healthcare workers and their increasing average age exacerbates the problems of recruitment and retention in developed countries. The migration of skilled healthcare workers from developing countries places additional strains on the scarce human resources in these countries.

A significant factor in this serious healthcare situation is the transmission of infectious diseases by accidental needle stick injury. In the U.S. and Canada, legislation is in place mandating the use of medical devices with engineered safety features in an attempt to reduce needle stick injuries. This has led to the development of a number of safety syringes and needle related devices with different modes of operation, protecting used needles immediately after use, disposing of them in safety containers and eliminating sharp metal needles where possible. The subject cannula can replace the use of sharp metal needles for many clinical and pharmaceutical applications.

Medical device safety initiatives have in some cases been very successful but in others have not been as effective as anticipated or required. This may result from a number of factors including resistance to the need to change behavior or learn new techniques. In addition, the design of some safety devices has been too complex, not user friendly, or viewed by some as too expensive to adopt. Other factors resulting in failure of acceptance include devices which are difficult to train healthcare workers to use, require unusual dexterity or lack sound ergonomic design. On this background, simple, intuitive safety devices requiring minimal expertise for use and short or no training and learning cycles are most likely to be successfully adopted and result in significant reduction in needle stick injuries.

A number of designs of blunt cannula, pre-slit elastomeric membranes, e.g., septums, or Luer activated valves have been developed in an attempt to allow transfer of fluids without the use of sharp metal needles in situations where injections through the skin are not required. These would include fluid transfer or drug mixing, accessing ports of intravenous administration sets, i.e., IV lines, withdrawing fluid from medication vials and adding medication to intravenous solution bags. For example, at present, healthcare workers including pharmacists frequently aspirate solutions from medication vials for mixing or administering solutions. This process usually involves a large bore, sharp metal needle and a number of procedural steps. Attention must be paid to ensure sterility, accuracy and as much safety as possible. The number of steps involved can be quite large and the time taken to implement the steps significant. Plastic needles or cannula which are sufficiently sharp to penetrate the unsupported membrane of a medication vial stopper yet sufficiently blunt to prevent the easy penetration of a supported latex or rubber membrane such as a rubber glove worn on the hand are known. For example, see U.S. Pat. Nos. 6,616,632 and 6,394,979.

More specifically, it is normal practice for healthcare workers while removing liquids from a vial to insert a standard sharp metal needle attached to a syringe through the stopper of the vial, i.e., the elastomeric membrane, invert the vial and withdraw the solution into the syringe. During this process, the healthcare worker carefully positions the tip of the needle, i.e., the external opening of the lumen of the needle just inside the vial, i.e., close to or directly adjacent the inner surface of the elastomeric membrane of the vial. The usual procedure to withdraw all or most of the fluid from the vial is to invert the vial ensuring pooling of the fluid contents downwards to the neck of the vial. With the tip of the hollow needle positioned just inside the vial as described, complete withdrawal of the fluid contents can be achieved. This procedure also minimizes the amount of unwanted air which is drawn into the syringe and which subsequently has to be expelled.

Moreover, the positioning of the needle tip may require repeated fine adjustments to ensure that the opening of the needle is at an optimal position. A bright and shiny metal needle can be relatively difficult to see because of the stainless steel material and reflections on both the needle and the curved surfaces of the neck of the vial or port, particularly if it is glass. Where the stopper and neck of the vial meet, i.e., where the needle tip is optimally positioned, the increased curvature of the glass or plastic vial neck may add to the difficulty of visualizing the needle tip.

Recapping a sharp metal needle is a procedure which is frequently associated with accidental needle stick injuries. A sharp metal needle is frequently removed from the syringe after it has been filled and usually requires recapping to accomplish this.

In addition, any inappropriate disposal of the sharp metal needle or even the understandable complete failure to dispose of a used contaminated needle in the chaotic clinical situation such as emergency cardio pulmonary resuscitation (CPR), may result in an accidental needle stick injury to waste disposal or janitorial staff.

Generally in clinical use the sharp metal needle used for fluid transfer, having no retention mechanism, may easily and inadvertently slip out of a medication vial or IV line access port and expose the sharp tip thus creating the potential for accidental needle stick injuries.

### BRIEF SUMMARY OF THE INVENTION

In a one embodiment of the present invention, there is provided a cannula for transferring fluid relative to a vial or intravenous port or container having an elastomeric or other sealing membrane, comprising: a cannula body having first and second opposite ends; the first end terminating in a solid, i.e., non-hollow tip for penetrating the elastomeric membrane; the body having a passage opening through the second end and extending within the cannula body towards the first end, the passage opening through at least one port through a side surface of the cannula body thereby to enable flow of fluid along the passage and between the opening through the second end and the side port; indicia on the cannula body between the second end and the side port representing a predetermined distance substantially corresponding to the extent of penetration of the side port of the cannula body through the membrane necessary to locate at least a portion of the side port on the opposite side of the membrane from the second end and directly adjacent the membrane.

In another embodiment of the present invention, there is provided a cannula for transferring fluid relative to a vial or intravenous port having an elastomeric membrane, comprising: a cannula body having first and second opposite ends; the first end terminating in a tip for penetrating the elastomeric membrane; the body having a passage opening through the second end and extending within the cannula body towards the first end, the passage opening through at least one port through a side surface of the cannula body thereby to enable flow of fluid along the passage and between the opening through the second end and the side port that is a shortened fluid path when compared to a cannula with an opening at the tip; a stop carried by the cannula body for engaging the membrane upon penetration of a portion of the cannula body through the membrane to locate at least a portion of the side port on the opposite side of the membrane from the second end.

It is also a feature of the invention that a cannula is enabled for Luer slip or Luer fit connections, even when used with syringe barrels designed for Luer Lok® connections. For purposes of this application, it will be understood that the terms “Luer fit” or “Luer slip” are used interchangeably herein as typically used in the art to refer to interfitting male/female cone surfaces with no other connection means.

The terms Luer Lok® or Luer lock are also used interchangeably to refer to similar connections but with the addition of threads and lugs that engage the threads.

In addition, reference is made often herein to an “elastomeric membrane,” a “septum” and a “vial stopper.” While the term “septum” is typically used in connection with an IV line, all of the terms are used interchangeably herein to refer to the component penetrated by the cannula.

It is also a feature of the invention that, as the cannula is removed from a vial, horizontally-oriented, elliptical or oval-shaped ports in the cannula body are occluded by the flexible vial stopper, thereby preventing leakage during the removal.

The invention will now be described in detail, in connection with the drawings identified below.

### BRIEF DESCRIPTION OF THE DRAWINGS

FIG. 1 is a schematic side elevational view of a standard syringe including a representation of an embodiment of a cannula and a cap according to a preferred embodiment of the present invention;

FIG. 2 is a side elevational view of the syringe with the cap removed and cannula inserted into a standard medication vial;

FIG. 3A is an enlarged side elevational view and of the cannula hereof;

FIG. 3B is a view similar to FIG. 3A illustrating the cannula penetrating the septum of a medication vial;

FIG. 4 is a perspective view of the one-piece cannula with finger flanges, cannula retention features and a cannula Luer connector cap poised below the medication vial;

FIG. 5 is a schematic view of the transfer cannula according to a further form of the present invention;

FIG. 6 is a view similar to FIG. 5 illustrating a further form of cannula hereof with cutting edges to cut through an elastomeric membrane;

FIGS. 7-9 illustrate further embodiments of the cannula hereof; and

FIG. 10 is a schematic side elevation of another cannula with a side adapted to be sealed by a vial stopper during removal of the cannula from the vial.

### DETAILED DESCRIPTION OF THE INVENTION

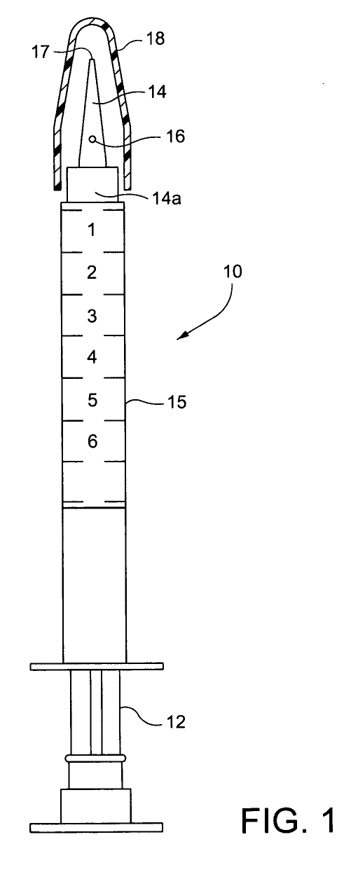

Referring to the drawings, particularly FIG. 1, a syringe generally designated 10 is illustrated including a plunger 12 and an integral cannula manufactured as a syringe barrel extension shown as 14 and 14A with a lumen opening or port 16 through a side surface of the integral cannula in communication with a passage, preferably axial through the cannula, in turn an extension of and therefore in communication with the interior of the syringe barrel 15. The tip 17 of the cannula 14 is semi-sharp, enabling the cannula 14 for penetration through the septum, i.e., an elastomeric membrane stopper of a vial, or a septum (possibly pre-slit) of an IV line access port. The relative bluntness of the tip 17 generally precludes penetration of the skin or of a protective glove as often worn by an individual using the syringe 10. The solid tip ensures that the insertion of the cannula through a membrane does not core the membrane or produce unwanted particles during insertion. The side opening port inevitably reduces the length of the fluid path compared to a passage which opens at the tip. In addition as the passage does not traverse the narrow or narrowing portion of the cannula at the tip a relatively large diameter of the passage is permitted. These last two parameters reduce the resistance to fluid flow through the cannula assisting filling and helping to reduce unwanted air entry and bubble formation within the syringe. Optionally, lubricity of the cannula may be enhanced by coating with a suitable commercially available polymer material, such as Parolene. In FIG. 1, a guard 18 is illustrated overlying the cannula 14 and is secured to the barrel 15.

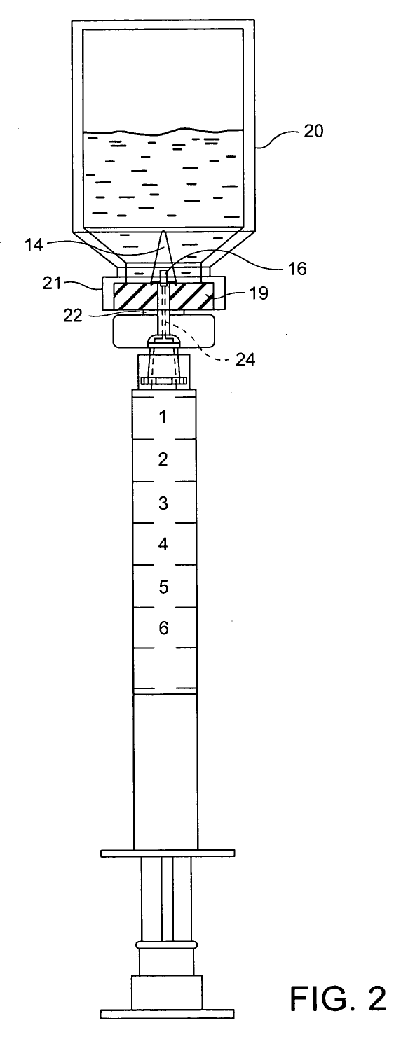

In FIG. 2, a removable, Luer-connecting cannula is shown affixed to the Luer connector of a syringe and the cannula tip 14 is illustrated inserted through the elastomeric membrane or stopper 19 of a vial, e.g., a medication vial 20. The opening port 16 through the side surface of the cannula lies just within the vial adjacent the inner surface of the elastomeric membrane 19. It will be appreciated that most medication vials have stoppers 21 with septums, i.e., elastomeric membranes 19 of similar thickness. Accordingly, a stop 22 may be located along the side of the cannula 14 for locating the port 16 directly and closely adjacent the inside surface of the septum. The stop may also provide compression of the membrane against the portion of the cannula on the other side of the membrane and thereby provide some rotational and axial stability of the cannula at the desired predetermined optimal position. This will improve the ease and speed of removing the cannula vial combination from the luer connector at the front of a filled syringe either Luer fit or Luer Lok®. It will also be appreciated that more than one opening 16 may be provided, e.g., four openings at 90 degrees apart or any other number of openings at various circumferential or axial spacings and that various configurations of the ports are possible. The openings may be at right angles to the axial lumen or inclined in a forward or rearward direction relative to the axial passage 24 through the cannula 14 communicating between the port 16 and the interior of the syringe barrel. For example, a forward opening for port 16 would be appropriate upon withdrawal of the cannula to preclude excess fluid from streaming toward the face of the user if it is anticipated that fluid will be expressed through the cannula while it is not inserted into another container or port.

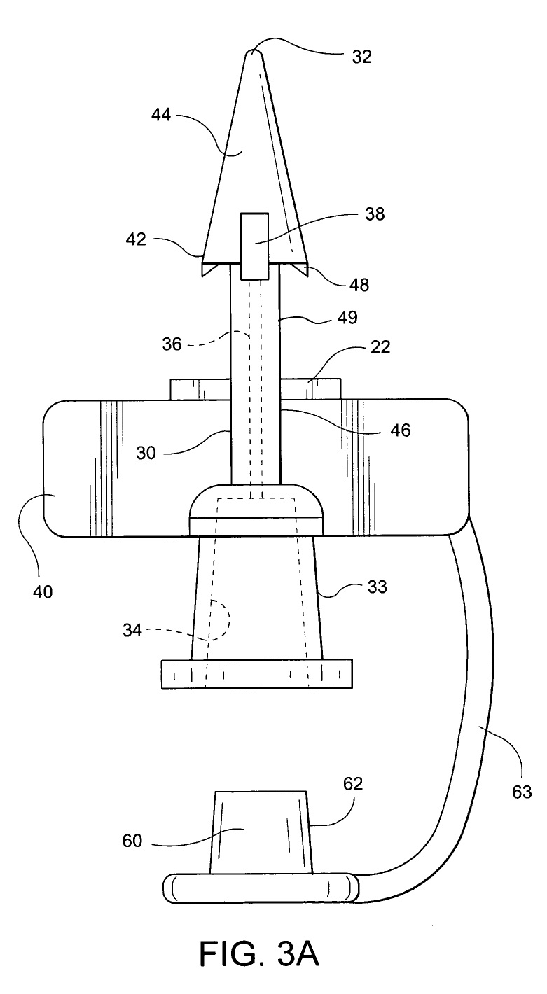

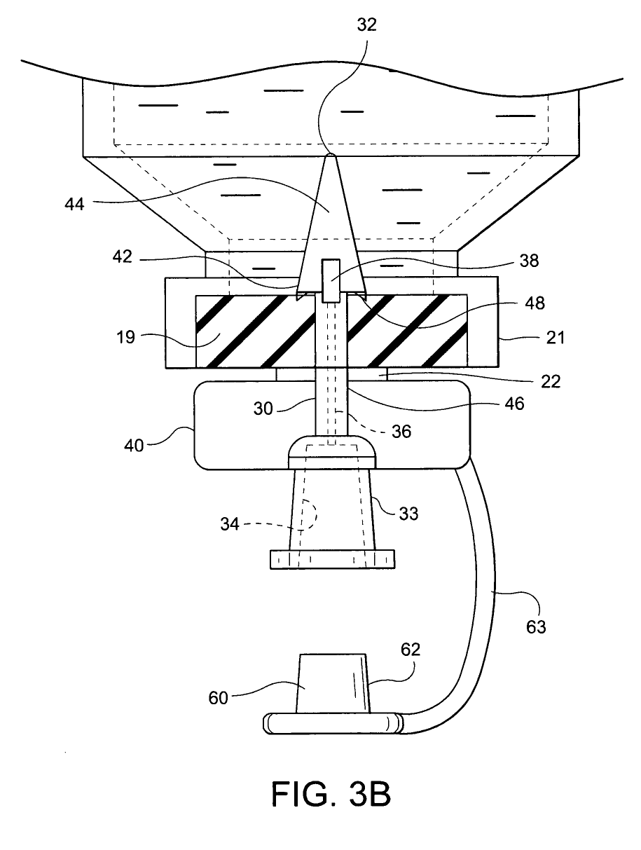

Referring to FIGS. 3A and 3B, the preferred form of a removable cannula 14 of FIG. 2 is illustrated with greater particularity including a cannula body 30 terminating at one end in a cannula tip 32 similar to tip 17. The proximal or second end of the cannula body 30 terminates in a hub 33 having a generally frusto-conical shaped recess 34, e.g., a lower opening for receiving a complementary Luer shaped and dimensioned fitting, e.g., a frustoconical recess for receiving a complementary shaped conical male fitting on the end of a syringe barrel. The frustoconical recess 34 opens into a passage 36 extending partway through the annular body terminating in one or more side openings or ports 38 along the side surface of the cannula body 30. Finger flanges 40 are provided adjacent the second end or base of the cannula 30 to facilitate application and removal of the cannula relative to a Luer fit of a syringe male Luer connector as well as rotational, and axial stabilization of the cannula fitted onto the syringe after or during penetration of the cannula tip through the elastomeric membrane 19 of a vial or IV port. Preferably, the finger flanges comprise a pair of lateral projections on the cannula body 30 at about 180° apart from one another.

To enhance or diminish the mechanical strength of the attachment of the cannula to a standard syringe with a male Luer fitting conical projection, the dimensions or configuration or frusto-conical shaped recess 34 may be varied and not conform precisely to the standard Luer dimensions or configurations. The flexible nature of the plastic material of the syringe and cannula Luer connection would be expected to allow the dimensionally mismatching Luer connection to continue to provide an adequate fluid seal. The tab projections (FIG. 8 at 68, FIG. 6 at 64) which would normally engage the Luer threads (FIG. 6 at 65) of a Luer Lok® connection may be present or absent similarly effecting the mechanical strength of the Luer connection and the ability to easily and rapidly disconnect the two. Since the introduction of Luer Lok® connections all commonly available syringe interfacing devices have tabs to engage the Luer threads and therefore required significant rotation to connect or disconnect with a Luer Lok® syringe or other device. The secure connection was required as in almost all instances there was a significant positive pressure generated during injection which would tend to disrupt a standard Luer slip connection (which does not have the external threads required to interact with the Luer Lok® syringe). In at least one of the embodiments the cannula is intended only for aspiration, i.e., to fill or partially fill the syringe after which the cannula will be removed to allow the syringe to interface with another Luer Lok® device such as a Luer Activated Valve (LAV) for injection. As this particular embodiment would not be used for injection, the strong and potentially disruptive pressure generated in the syringe during injection will not be applied to the cannula syringe connection. Hence the rotational Luer Lok® connection is not only not necessary but more time consuming and slightly less ergonomically advantageous than the straight pull off present in the described embodiment (absent the ovoid Luer thread engaging tabs). In summary, omitting the tabs will allow the removal of the cannula from either standard Luer or Luer Lok® syringes without the need for rotation and unthreading of the cannula from the syringe (i.e., Universal Luer slip).

In this preferred embodiment, the cannula body 30 includes a head or penetration portion 44 which tapers from the tip 32 to an intermediate laterally enlarged transition portion 42 of the cannula body 30 and then to a laterally diminished portion or waist 49. While the taper from the tip 32 to waist portion 42 is about an axis of symmetry and forms a conical surface of revolution about the axis, it will be appreciated that the penetrating portion 44 of cannula body 30 may have other configurations, such as a concave surface of revolution. For example, the penetration portion 44 may be asymmetrical with respect to an axis between opposite ends of the body 30, may be cylindrical or oval at any cross-sectional configuration through the penetration portion 44 or may comprise ridges following the generally conical surface with concave recesses between adjacent ridges about the body 30. The waist portion 49 may likewise have the same cross-sectional configuration at the juncture of the waist portion 49 and the penetrating portion 44, i.e., cylindrical, oval, multi-channeled or the like.

In this embodiment, central portions 46 and 49 of the cannula body between the waist portion 42 and the proximal end (the lower end of body 30 in FIG. 3) may have the same or a different cross-section than the cross-section of the penetrating portion 44. For example, whereas the penetrating portion 44 may have a cylindrical cross-sectional configuration at any length therealong, the central portions 46 and 49 may have a cylindrical configuration or an oval configuration or any other type of cross-sectional configuration which will accept a seal when encompassed by the elastomeric membrane upon penetration of the cannula body through the membrane. As illustrated, the passage 36 extends from the tapered internal recess 34 to one or more openings or ports 38 through the side surface of the cannula body 30. While each side port 38 may be cylindrical and may open 90° relative to the length direction of the cannula body 30, preferably each side port 38 is elongated in a direction towards the opposite ends of the cannula body and lies in the region of the waist portion 42. It will be understood, however, that in order to prevent leakage when the cannula is removed from a vial, the axial length component of the opening slot 38 should be smaller than the axial thickness of the vial stopper or membrane, as shown for example in FIG. 3B. Additionally, the undersurface of the transitional portion 42 may include anti-rotation features to preclude relative rotation of the cannula body and elastomeric septum when the cannula has penetrated the septum and lies in fluid communication with the vial or IV line. In one form, the anti-rotation feature includes a plurality of flanges 48 projecting toward the second or proximal end of the cannula body. Alternatively, a series of projections, e.g., dimples, ridges or simply a roughened surface along the underside of transitional portion 42 suffices to afford contact with the inside face of the septum when the cannula is engaged with the septum to preclude relative rotation between the cannula and the vial or IV port. Opposite the underside of the transitional portion 42 are rotational stops 22 which project in a direction toward the first end of the cannula 10. The stops 22 engage the outer surface of the elastomeric membrane to preclude or inhibit relative rotation between the cannula and vial or IV port and enhance axial stability. Stops 22 preferably terminate radially inwardly of the metal cap normally found on a medication vial. In this manner, the rotational stops 22 project beyond and into engagement with the outer surface of the elastomeric membrane to preclude or inhibit relative rotation between the cannula and vial before the margins of the finger flanges 40 engage the metal cap of the vial or IV port which otherwise would permit slippage between the cannula and the vial or IV port. It can be appreciated that this described enhanced axial and rotational stability affords improved ergonomics for separating the filled syringe from the vial stopper/cannula combination at the end of filling. The winged finger flanges also assist in this function.

Additionally as illustrated in FIGS. 3A and 3B, there is provided a closure cap 60 for the proximal or second end of the cannula, e.g., female Luer connection lumen. Preferably, the closure cap 60 is attached to the cannula by a living hinge connected to the cannula body. The closure cap includes a frusto-conical surface 62 complementary to the interior frusto-conical surface 34 at the second end of the cannula. Thus, by inserting the cap 60 into the frusto-conical opening 34, at the proximal end of the cannula, the cap seals the opening at the proximal end against transfer of fluid in either direction along passage 36. Cap 60 is preferably secured to the cannula 30 by a tether 63. However, cap 60 may be provided separate from the cannula 30, and removeably attached by any suitable means to the cannula. For example, the tether 63 could be provided with a lug snap-fit into an aperture in one of the finger flanges 40.

From the foregoing, it will be appreciated that the cannula 30 is a single unitary or integral cannula (with or without cap 60) formed of a plastic material. For example, polypropylene, ABS, or polycarbonate materials may be utilized to mold the cannula with or without a syringe barrel cannula extension although it will be appreciated that other materials may be utilized. Suffice to say that the integral one piece nature of the cannula facilitates its manufacture at low cost. Similarly, where the cannula is integral with the barrel or barrel extension the single unit manufacture is cost efficient.

The removable cannula may be grasped by the fingers of the healthcare worker about the finger flanges 40 and thereby readily manipulated for placement on a syringe. The cannula tip 32 is then brought into engagement with and penetrates through the elastomeric membrane 19 of the vial or IV port. Typically, medication vials do not have a slit similar to slits in some IV ports. Accordingly, the cannula body may be advanced with sufficient force to penetrate through the elastomeric membrane of the medication vial or pass through the pre-slit or other membrane of an IV port. The cannula is advanced until the elastomeric membrane 19 registers in the waist between the transitional portion 42 and the rotational stop 22. Because elastomeric membranes used in the vast majority of vials and IV ports have a substantially common thickness, the distance between the underside of the waist portion 42 and the surface of rotational stop 22 represents a pre-determined distance substantially corresponding to the extent of penetration of the side port 38 of the cannula body 30 through the elastomeric membrane or vial stopper necessary to locate at least a portion of the side port on the opposite side of and directly adjacent the membrane. This dimensional relationship thus enables the cannula body to be thrust through the membrane until stopped by the engagement of the stop 22 along the outside surface of the membrane or the engagement of the finger flanges 40 about the margin of the medication vial or IV port. Also, the side port 38 is located relative to the stop 22 such that the port 38 is located on the opposite side of the membrane from stop 22 directly adjacent the membrane. In this manner and particularly for use with medication vials which are inverted to withdraw fluid from the vial into a syringe, the side port is positioned to enable withdrawal of substantially the entire contents of the medication vial. That is, the distance between the stops 22 or the finger flanges 40 (FIG. 3B) and the side ports 38 corresponds to indicia on the cannula body representing a predetermined distance corresponding to the extent of penetration of the side port through the membrane necessary to locate a portion of the side port on the opposite side of the membrane from the proximal end of the cannula and directly adjacent the membrane. Thus, the cannula is automatically self-positioned such that the side port or ports 38 lie at a predetermined location relative to the membrane or stopper 19 and adjacent the inside surface of the stopper. As noted, the side port or ports 38 will lie just distal to the inside surface of the vial stopper to facilitate withdrawal of the entire contents of the vial. Moreover, the self positioning of the cannula is repeatable when fully inserted through the vial membrane and the positioning stop of the cannula comes to rest in contact with the membrane. This allows rapid, easy and predictable cannula positioning without visual reference, i.e., even if the vial is opaque or semi-opaque or in circumstances of poorer lighting or where visual positioning is difficult. This self or automatic positioning feature removes the necessity to manipulate the cannula once it has fully penetrated the membrane.

The undersurface of the transitional portion 42 in conjunction with the stop 22 also provide stabilization and retention features. For example, the location of the membrane within the slot or groove provides axial stability and the projections 48, with or without the stop 22 also provide rotational stability thereby maintaining optimal positioning of the cannula relative to the membrane with respect to various functions such as the ideal fluid withdrawal position or stability about axial or rotational axes during removal of the syringe from the cannula. The accuracy of the automatic positioning of the cannula and the port 38 ensures optimal emptying of the vial and tends to reduce aspiration of unwanted air as the fluid is withdrawn into the syringe. The side port(s) 38 will remain in communication with the fluid in the neck of the inverted vial until virtually all of the fluid contents are removed or the dose required is withdrawn. This has significant benefits in saving the time and effort required to position the cannula, fill a syringe and then perform the usual necessary removal of unwanted air from the syringe. Moreover, the elongation of the one or more ports 38 accommodates potential variations in the thickness of the membrane such that alignment of at least portions of each of the ports 38 with the interior of the inverted vial is assured. Further, the diameter of the fluid passage 36 is not dependent upon the diameter of the cannula tip 32 as it otherwise would be in the case of a sharp metal needle requiring the passage to terminate within the tip. As a consequence, the diameter of the passage 36 can be significantly greater than that of a standard hollow boreneedle of similar tip dimension. Also, the lateral port 38 may have a similar or larger flow area than the passage 36. These features reduce significantly the resistance to fluid flow and the forces required to fill or empty the syringe with the cannula attached to the syringe. The reduced pressure requirements during filling will assist in preventing leakage of air past the stopper on the end of the plunger or through the seal at the end of the syringe which may be necessary in a safety syringe with a retractable needle. The increased flow rate consequent on the reduced resistance described will limit the partial vacuum which is typically developed on withdrawal of the plunger and will reduce the likelihood of the dissolved gas present in the aspirated fluid from coming out of solution to form bubbles. The reduced propensity for air bubble formation assist the end user in clearing all bubbles from the syringe prior to administering the injection improving speed and accuracy of dose delivery. It will be appreciated that the solid semi-sharp cannula tip and the side external lumen port or ports 38 proximal to the distal tip render intentional or accidental use of the cannula for penetrating the individual's skin and injection of fluid highly unlikely, if not impossible. This will limit the inadvertent or attempted misuse of the cannula which has been described with tip opening cannulae.

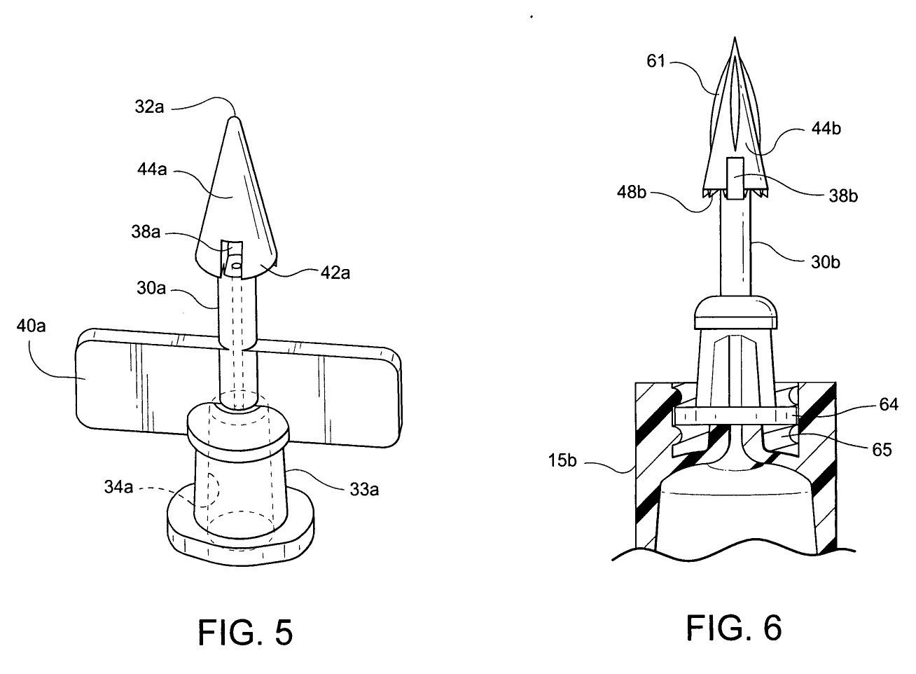

In FIG. 5, there is illustrated another preferred embodiment of the cannula hereof wherein like reference numerals are applied to like parts as in the previous embodiment followed by the letter suffix “a”. Cannula 30a is a similar to the cannula 30 of FIGS. 3A and 3B except that the stops 22 have been omitted. Preferably, the distance between the waist portion 42a and the edges of the finger flanges 40a correspond to the thickness of the septum 19 thereby locating the ports 38a directly adjacent the inside face of the septum 19. In this drawing Figure, the ports 38a are illustrated 180° apart.

Referring to FIG. 6, there is illustrated a further embodiment of the present invention wherein like reference numerals are applied to like parts followed by the letter suffix “b.” In this embodiment, the penetrating portion 44b of the cannula 30b includes a plurality of ribs 61 circumferentially spaced about the cannula body and proud of the tapered surface thereof. The ribs enable the cannula to open a sufficient passage in a previously unpenetrated elastomeric membrane 19 to allow the remainder of the cannula to pass through the membrane exposing the port 38b directly adjacent to the inside face of the membrane. Also, the ribs 61 enable a cap 60 to frictionally interface with the ribs 61 to facilitate retention of the cap on the cannula body 30b. As in the prior embodiments, it will be appreciated that the cross-section of the cannula penetration portion 44b need not be annular and that the ribs 61 may take other forms and numbers thereof than illustrated. Also, the finger flanges may be omitted from this embodiment (as shown) as well as in the other embodiments.

Also as illustrated in FIG. 6, the cannula 30b includes a plurality of anti-rotation projections 48b extending in a direction toward the second end of the cannula for engaging the inside surface of the membrane. As previously noted, those projections 48b inhibit relative rotation between the membrane and the cannula.

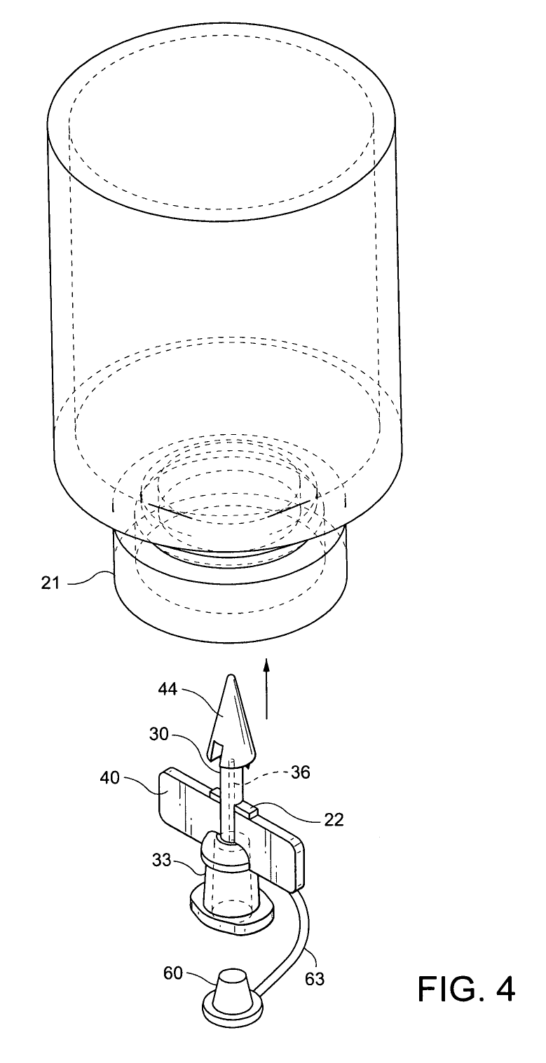

FIG. 6 also illustrates how a generally ovoid-shaped flange 64 at the base of the cannula body 30b engages the Luer threads 65 in the syringe barrel 15b. Similar flanges are also shown at 66 (FIGS. 3A, 3B, 4), 68 (FIG. 5), 70 (FIG. 67), 72 (FIG. 8) and 74 (FIG. 9). The use of an ovoid-shaped flange to engage Luer threads to establish a Luer lock connection is well known. On the other hand, it may be beneficial in some instances to omit the flange, thus leaving only a smooth, circular edge or end on the hubs 33, 33a, 33b, 33c and 33d. The circular end absent the flange would be too small to engage the Luer threads as best appreciated from FIGS. 6 and 8. However, the male Luer cone (see cone 73 in FIGS. 6 and 8) could still be utilized to engage a respective recess 34-34d in an alternative Luer slip connection that would not require rotation for removal. Thus, the use of hubs without ovoid-shaped flanges could be used to create a Luer slip (non-threaded) connection between the cannula and the syringe even when used with syringe barrels provided with threads for Luer locks. After syringe filling it may be desirable to attach the syringe to other components securely using Luer lock connections. The removal of these flanges allows both safe and watertight Luer fit connections with either Luer Lok® connections or Luer fit connections, a function not permitted by conventional needle and cannula fittings.

In this regard, while the threads on the syringe barrel may be required for establishing a Luer lock connection with a standard needle or IV line after filling, a Luer fit or slip connection with the cannula for purposes of filling as described herein may be sufficient.

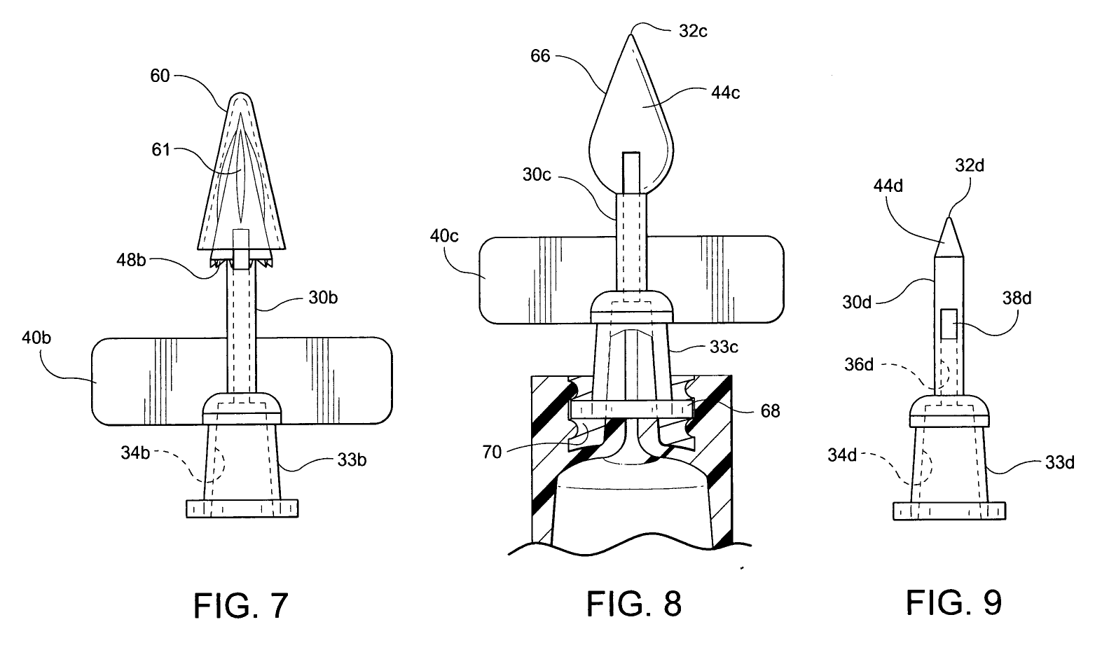

FIG. 7 illustrates the embodiment of FIG. 6 with the finger flanges 40b carried by the cannula body 30b, and with a cap 60 In use, the healthcare worker simply removes the guard 18 (see FIG. 1), engages the cannula against and penetrates the membrane, withdraws fluid from the medication vial and in one movement, in instances where the cannula and syringe have a Luer fit, may pull the syringe from the cannula leaving the cannula in the empty vial. If the vial is not empty, either to prevent spillage or maintain sterility for future additional aspiration of contents, the cap 60 can be applied to the open Luer end of the cannula while it is still inserted through the membrane. Where the cannula is integral with the syringe barrel the steps of affixing the cannula to the Luer connection are eliminated. Alternatively, the finger flanges 40b facilitate removal of the cannula from an unthreaded Luer slip fit on the barrel end of the syringe or facilitate threading of the cannula onto the syringe when the cannula is used with a threaded Luer lock fit on the syringe.

Referring to FIG. 8 wherein like reference numerals apply to like parts followed by the letter suffix “c,” the cannula 30c has a penetration portion 44c having a bulbous or convex outer surface 66 terminating in a blunt or semi-sharp tip 32c. Also, at the proximal or second end of the cannula body 30c, the proximal end terminates at the ovoid-shaped flange 72 forming part of a standard Luer lock for engaging the internal threads 70 the end of the syringe barrel.

Referring to FIG. 9, wherein like reference numerals are applied to like parts as in preceding embodiments followed by the suffix “d,” the cannula body 30d includes an intermediate body portion 30d between penetration portion 44d and hub 33d which preferably has a constant cross-sectional area. The intermediate portion may be cylindrical in cross-section but other cross-sections may be provided such as an oval cross-section. The port or ports 38d open through the sides of the intermediate section and, as in prior embodiments, the side ports 38d are located from the upper end of the hub 33d a distance corresponding to the width of the membrane 19. Thus, upon penetration of the cannula body 30d through the septum or membrane 19, the ports 38d will be located directly adjacent the inside face of the septum 19 and further penetration of the cannula body through the septum will be prevented by the abutment of the upper edge of the hub 33d against the outerface of the septum 19. As in all previous embodiments, the cannula body 30d terminates at its distal end in a penetration portion 44d having a semi-sharp tip 32d. It can be appreciated that this embodiment has the self positioning features seen in other embodiments but lacks the axial or rotational stabilizing features.

To enhance the speed of syringe filling, significantly strong forces may be used to rapidly withdraw the plunger and create a partial vacuum in the syringe. This technique requiring application of an axial force to the plunger may at times result in the unexpected displacement of the previously accurately positioned cannula tip through the vial stopper. The disclosed cannula in some embodiments reduces significantly this possibility because of the cooperation of the engagement of the vial or port with the stops 22 or finger flanges 40 against the vial or port and the position and configuration of retention features such as 48B. Additional performance benefits reside in the positioning of the cannula relative to the vial or IV port, the relative fixation of the cannula through cooperation of the vial or IV port, stopper and cannula and the elimination of the need for visualization of the cannula tip when inserting the tip through the membrane. Further, the ease of completely emptying the vial and the reduction in inadvertent aspiration of air into the syringe are added performance benefits. The single piece integrally molded plastic cannula and the ability to manufacture it if so desired integral with a syringe barrel, may result in improved simplicity in use, packaging and manufacturing with resultant cost reduction. The ease of fully emptying the vial will enable as much of the contents of the vial as possible to be easily removed. Manufacturers aware of the current difficulty of fully emptying a vial will overfill with additional drug contents, often 10%, to ensure the vial will allow at least the nominal fluid volume to be aspirated using conventional needles. The side opening optimally self positioned cannula will enable the full contents including overfill to be aspirated, effectively reducing significantly the cost of each drug dose.

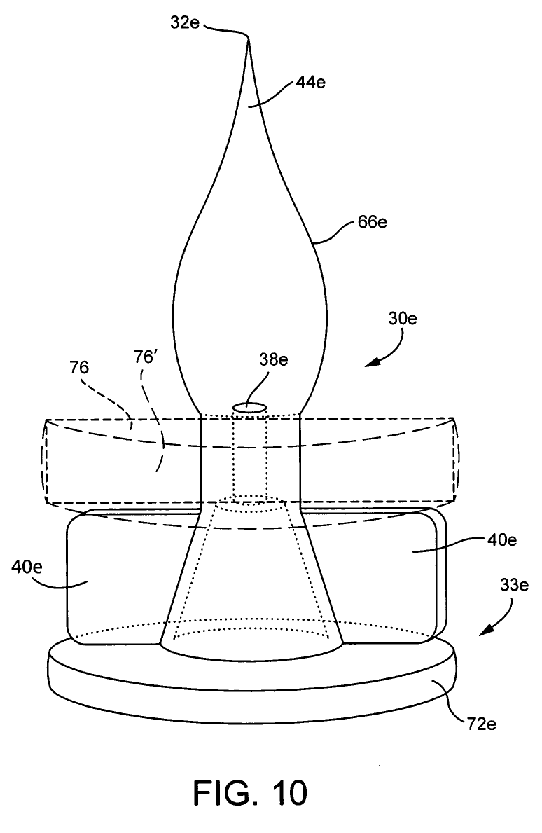

Referring now to FIG. 10, wherein like reference numerals are applied to like parts as in preceding embodiments followed by the suffix “e”, the cannula body 30e has a penetration portion 44e having a bulbous outer surface 66e terminating at a blunt or semi-sharp tip 32e. At the proximal or second end 33e of the cannula body 30e, there is formed a radial flange 72e, designed to establish a Luer lock connection with the threads of a syringe barrel (as shown, for example, in FIGS. 6 and 8).

One or more side ports 38e is formed in the cannula tip, just adjacent the bulbous outer surface 66e of the cannula. The one or more elongated ports 38e are generally horizontally oriented and rounded in shape, i.e., the ports have an oval or elliptical shape. A vial stopper 76 is shown in phantom, positioned as it would be when the cannula tip 30e is fully inserted within a vial, with finger flanges 40e serving as limit stops to the penetration of the cannula. This dimensional relationship, where the axial width of the opening 38e is considerably smaller than the thickness of the vial stopper or membrane 76, eliminates leakage during withdrawal of the cannula from the vial. In addition, as the cannula 30e is withdrawn from the vial, the flexible vial stopper or membrane 76 will bow in the direction of the syringe barrel (as shown in phantom at 76′), so as to conform to the base of the bulbous portion 66e as it is pulled through the stopper, with the flexible stopper surface thereby wiping across and sealing or occluding the port or ports 38e until the cannula is fully withdrawn from the vial.

Various performance features of the disclosed embodiments, while not all inclusives, may be summarized as follows:
The solid tip of the cannula is sufficiently sharp to penetrate and puncture drug vial stoppers.
The solid tip of the cannula is not sufficiently sharp to penetrate a latex or rubber glove worn on an individual's hand.
The solid cannula tip is relatively blunt (i.e., semi-sharp) and can penetrate skin only with considerable difficulty and force.
Reduced likelihood of accidental needle stick injury.
Reduced likelihood of disease transmission if a needle stick injury occurs because of the solid tip. (vs. hollow)
The cannula ports are of optimized configuration, distant from the cannula tip and lie at optimized positions for complete evacuation of fluid from the vial.
The vial stopper or IV port membrane captures the cannula within the stopper or membrane at reliably repeatable axial positions.
Suitability and compatibility for use with standard Luer fit or Luer lock with little or no training required for use.
One piece integral product manufactured at low cost using inexpensive materials.
May be manufactured integral with a syringe barrel.
Non-coring penetration, i.e., the solid tip, reducing likelihood of undesirable particulate formation and medication contamination.
Solid tip reduces likelihood of disease transmission in comparison with standard hollow tip metal needles.
Enhanced lubricity of the cannula by secondary treatment e.g., Paralene improves the ability to penetrated easily and fully a membrane
Enhanced performance in flow rate and pressure during use, since fluid passage is shorter and possibly wider as it is not dependent upon the diameter of an opening at the needle tip as in standard metal needles.
Diminished likelihood of air aspiration from the vial while filling the syringe.
Diminished partial Vacuum developed while filling the syringe and reduced likelihood of air leakage past the plunger stopper.
Diminished partial vacuum developed and reduced tendency for gas to come out of solution to form air bubbles within the syringe.
Improved visibility of cannula over similar sized needles.
Automatic optimal cannula positioning without need for visualization.
Features tending to cause either or both rotational and axial stability when the cannula has penetrated the vial or IV port membrane.
Reduced number of procedural steps during standard aspiration procedure.
A modified flange at the base of the cannula body permits the cannula to be used in a Luer slip connection even when the syringe barrel is designed for a Luer lock connection.
Elliptical or oval side ports on the cannula tip may be horizontally arranged so as to be sealed or occluded by the vial stopper upon withdrawal of the cannula from the vial.

It will be appreciated that the foregoing disclosure and features provide an aspiration/injection semi sharp cannula which enhances the safety and efficiency of the transfer of fluids and medication solutions and can be used with currently available standard equipment simplifying the transferal process resulting in time and cost savings and by eliminating in some cases the need for sharp metal needles improved safety.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A system for transferring fluid between a vial and a cannula, comprising:
a vial having a flexible stopper;
a cannula body having first and second opposite end;
said first end terminating in a solid tip for penetrating the flexible stopper; said body having a passage opening through said second end and extending within said cannula body towards said first end, said passage opening through at least one non axially oriented, port through a side surface of the cannula body thereby to enable flow of fluid along said passage and between the opening through said second end and said side port; and
wherein an axial width of said port is smaller than a thickness dimension of the flexible vial stopper through which the cannula tip is to be inserted.

2. A system according to claim 1, including a stop carried by said cannula body for abutting the flexible stopper and thus precluding further penetration of the cannula body past the stopper into the vial.

3. A system according to claim 1, wherein said passage extends axially at least in part through the cannula body.

4. A system according to claim 1, wherein said cannula body is generally elliptical in shape and substantially perpendicular to an axis along said cannula body between said opposite ends.

5. A system according to claim 1, wherein said cannula body is generally asymmetrical about an axis along the body between said opposite ends.

6. A system according to claim 1, wherein said solid tip is insufficiently sharp to easily penetrate an individual's skin or a protective glove.

7. A system according to claim 1, wherein said second end includes a frusto-conically tapered recess with sidewalls converging toward one another in a direction towards first end.

8. A system according to claim 7, wherein said second end terminates at a smooth, circular edge, enabling the cannula to engage a syringe barrel in a Luer-slip fit.

9. A system according to claim 1, wherein said second end includes a flange adapted to engage syringe barrel threads in a Luer lock fit.

10. A system according to claim 8 wherein the syringe barrel is provided with threads for a Luer lock fit with said cannula body.

11. A system according to claim 1, wherein said cannula body includes a lateral projection for engaging the opposite side of the flexible stopper to at least inhibit withdrawal of the cannula body from the flexible stopper.

12. A system according to claim 11, wherein said lateral projection includes a continuous flange about the cannula body and located along the cannula body between the side surface port and said second end.

13. A system according to claim 11, wherein said lateral projection includes at least one protuberance to at least inhibit rotation of the cannula body relative to the flexible stopper.

14. A system according to claim 1 and further comprising a cap adapted to close said second opposite end of said cannula body.

15. A cannula for transferring fluid relative to a vial or intravenous port having an elastomeric membrane, comprising:
a cannula body having first and second opposite ends;
said first end terminating in a solid tip for penetrating the elastomeric membrane;
said body having a passage opening through said second end and extending within said cannula body towards said first end, said passage opening through at least one port through a side surface of the cannula body thereby to enable flow of fluid along said passage and between the opening through said second end and said port;
a stop carried by said cannula body for engaging the membrane upon penetration of a portion of the cannula body through the membrane to locate at least a portion of the port on the opposite side of the membrane from the second end; and
wherein said port has an oval or elliptical shape and is substantially non axially oriented.

16. A cannula according to claim 15, wherein the side portion is elongated in a direction toward the opposite end of the cannula body.

17. A cannula according to claim 15, wherein said stop precludes further penetration of the cannula body portion through the membrane when the side port lies directly adjacent said opposite membrane side.

18. A cannula according to claim 15, wherein second end terminates at a smooth, circular edge enabling the second end of the cannula to engage a male Luer cone or a syringe barrel in a friction fit.

19. A cannula according to claim 18, wherein said second end includes a flange adapted to engage threads in a Luer lock fitting on a syringe barrel.

20. A cannula according to claim 15 and further comprising a cap adapted to close said second opposite end of said cannula body.

21. A pair of components connected to one another comprising a first component formed at one end with Luer lock threads surrounding a center cone; and a second component having a center recess at one end thereof in which said center cone is received, to establish a Luer slip connection between the components wherein said Luer lock threads are not engaged by said second component.

22. The pair of components of claim 21 wherein said first component comprises a cannula and said second component comprises a syringe barrel.

23. The pair of components of claim 22 wherein said cannula comprises:
a cannula body having first and second opposite ends;
said first end terminating in a solid tip; and
said cannula body having a passage opening through said second end and extending within said cannula body towards said first end, said passage opening through at least one non axially-oriented port through a side surface of the cannula body thereby to enable flow of fluid along said passage and between the opening through said second end and said side port.
